(19) United States
(12) Patent Application Publication
(10) Pub. No.: US 2008/0317324 A1
(43) Pub. Date: Dec. 25, 2008

(54) Method and Device for the Characterization of Cells, Clusters and/or Tissue

(76) Inventors: Markus Eblenkamp, Munchen (DE); Ulrich Steinseifer, Haag (DE); Erich Wintermantel, Kranzberg (DE)

Correspondence Address: ALLEMAN HALL MCCOY RUSSELL & TUTTLE LLP, 806 SW BROADWAY, SUITE 600, PORTLAND, OR, 97205-3335, US

(21) Appl. No.: 10/588,887

(22) PCT Filed: Feb. 10, 2005
(86) PCT No.: PCT/EP2005/001347

§ 371 Date: Sep. 10, 2008

(30) Foreign Application Priority Data
Feb. 11, 2004 (DE) 10 2004 006 781.3

(51) Int. Cl. C12M 1/34

(52) U.S. Cl. 382/133

(57) ABSTRACT

This invention relates to an apparatus for characterizing cells, cell aggregates and/or tissue with an analysis unit which includes means by which morphological parameters of cells, cell aggregates or tissue are detected and which furthermore includes means by which the detected morphological parameters are evaluated for the purpose of the objective morphological characterization of the cells, cell aggregates or tissue. The invention furthermore relates to a method for objectively characterizing cells, cell aggregates and/or tissue.

This invention relates to an apparatus and to a method for characterizing cells, cell aggregates and/or tissue.

As a central tool of regenerative medicine, methods of cellular therapy are highly important for future innovative methods of treatment, where cells are grown and possibly manipulated in vitro on an industrial scale in a standardized way. Furthermore, it is known to use cell cultures as test objects in the pharmaceutical industry for testing pharmaceuticals.

The appearance of a two- or three-dimensional cell culture has specific morphological features which are each dependent on the type of cells cultivated, both on a single-cell level and on the level of the total cell population. The cells differ, for instance, in their size and shape, in the size of the cell nucleus, in the granularity, the migratory activity, the spatial relations between the cells (disperse or coherent) or, in the case of three-dimensional cell cultures, in their spatial orientation.

If it is required to describe the cell culture or assess its quality, microscopic methods are chiefly used at present, in order to detect the characteristics of the cell culture. However, the morphological assessment of the cell population, which largely is performed “by hand” (not automatically), greatly depends on the knowledge and the experience of the examiner and therefore remains subjective. As compared to other methods for characterizing the cell population (e.g. methods of molecular biology, FACS analyses), the morphological assessment involves the advantages that it can be performed online without disturbing the growth of the cell population to be analysed, that individual cells of a population can be analysed, that it can be performed quickly, and finally that it is inexpensive.

In the following, a currently used method for growing adherent cells is illustrated by way of example. In several stages of the procedure, morphological assessments of the cell culture are necessary:

In a first step (isolation of the cells), the cells to be cultivated are extracted from their original tissue by enzymatic and mechanical methods and are transferred to corresponding culture vessels upon purification. In dependence on the properties of the original tissue (e.g. bone, cartilage, adipose tissue), different methods and protocols will be employed.

Upon isolation, the cells are incorporated in a nutrient solution (“medium”) in a further step (cultivation of the cells in vitro), are transferred to a cell culture vessel, and are cultivated in an incubator at 37° C. and 100% humidity. Apart from water, the nutrient solution comprises a mixture of salts, nutrients, vitamins as well as growth and differentiation factors, whose composition is specific for the type of cells to be cultivated. The necessary media are only available to a certain extent and generally are mixed together personally in the laboratory. The medium is replaced at regular intervals by sucking off the old medium and adding a new one. In some cell cultures it is necessary to add various media with different compositions in accordance with a defined protocol in the course of the cultivation. Since adherent cell cultures grow on the surface of the cell culture vessel, it is required to periodically check by microscopy whether there is still sufficient room for growth on the surface (“degree of confluence”). If this no longer is the case, the cells must be distributed over several new cell culture vessels (“passaging”).

Passaging involves the step of detaching the adherent cells from the surface of the culture vessel by enzymatic or mechanical methods. The cells are purified by centrifugation, subsequently counted, and spread in new culture vessels in a defined quantity.

Apart from determining the degree of confluence, the quality of the cell population is assessed at regular intervals (analyses during cultivation). Particular attention is directed to a typical morphology of the cells, to the growth behavior as well as to possibly existing contaminations by microorganisms or undesired cell populations. The analyses are carried out in the form of an assessment by microscopy. The same can be applied online, but requires much personnel. In addition, the assessment by microscopy requires a morphologically trained eye and remains subjective in some fields (in particular in the assessment of the typical cell morphology).

It is the object of the invention to provide an apparatus and a method by means of which cells, cell aggregates and/or tissue can objectively be characterized in terms of morphology.

This object is solved by an apparatus with the features as claimed in claim 1, by an apparatus with the features as claimed in claim 7, and by a method with the features as claimed in claim 10.

The apparatus of the invention comprises an analysis unit which includes means for detecting morphological parameters of cells, cell aggregates or tissues. Furthermore, the apparatus includes means for evaluating the detected parameters for the purpose of the objective morphological characterization of the cells, cell aggregates or tissues. An essential idea of the apparatus or the method of the invention consists in describing the morphological image of the cell culture or of individual cells, preferably by a multitude of morphometric data. In this way, morphometric values can be obtained, which are characteristic for the respective cell culture or the respective individual cells (morphometric fingerprinting). In contrast to a figurative detection of the morphology, the morphometric fingerprint is objectified. The analysis unit of the invention preferably includes e.g. a software-assisted image-forming unit, such as a microscope, and a software-assisted image analysis unit, the image analysis unit including integrated expert knowledge, in order to be able to perform the evaluation of the detected parameters or a proper characterization.

The analysis unit preferably is configured such that the degree of confluence, the cell morphology as a measure for the quality of the cell culture, the proliferation behavior, the contamination with microorganisms or other cell populations and/or the cell differentiation can be determined and be evaluated. It is conceivable, for instance, to detect and then evaluate the cell size, the cell shape, the size of the cell nucleus, the granularity, the migratory activity, the growth behavior, the spatial relation between the cells (disperse, coherent) or, in the case of three-dimensional cell cultures, also the spatial orientation of the cells.

It is possible in principle to perform an evaluation of the parameters detected by forming indices which suitably relate the detected values to each other. It is conceivable, for instance, to form a quotient from the circumference of the cells and from four times the value of the square root of the cell area as an object index. This index can for instance be used to distinguish a fibroblast culture from an endothelial cell culture. In principle, any indices can be formed from the detected parameters, which are sufficiently distinctive as regards the characterization of cells, cell aggregates or tissue.

In accordance with another aspect of the invention it is provided that the analysis unit includes means for the statistical evaluation of the detected parameters. In such an embodiment of the invention, a statistic can be prepared from morphometric values which are characteristic for the respective cell culture or the respective individual cell. The statistically determined parameters can, for instance, be the mean value, the standard deviation, and the median of the morphological values. The statistical evaluation can relate to the directly detected parameters, such as the size of the cells, or also to the above-mentioned indices, which can suitably be formed from the detected values, as far as this is helpful for the characterization.

In accordance with another aspect of the invention, there is provided a database with reference parameters typical for the cells, cell aggregates or tissue. Furthermore, it is provided that the apparatus includes comparator means, by means of which the detected parameters can be compared with the reference parameters. On the basis of this comparison, a statement as to the cell population or the type and shape of the cell aggregate or tissue can be made in an automated and objectified way. It is possible that experts build up a database in which the morphometric data for reference cultures or cells are present. By comparing the values of a cell culture to be examined with those from the file, expert knowledge thus can indirectly be integrated in the morphological examination, and the morphological description of the cell culture to be examined can be objectified. By comparing statistically described morphometric characteristics of the cell culture to be examined with those from a database, the quality of the cell culture can be assessed without the presence of an expert. Similarly, the database can also be used for identifying individual structures unknown to the examiner (e.g. sedimentation, subcellular structures) as well as for tracing morphologically describable phenomena (screening for microbial contaminations).

The method and the apparatus of the invention can perform an evaluation by means of directly detected sizes, such as the cell size, or by means of the above-mentioned indices, which can be formed in a suitable way.

In accordance with another aspect of the invention it is provided that the analysis unit includes means by which adjacent pixels of the detected image with similar brightness values are combined to one image object. As the phase-contrast images virtually reveal the morphological structures (e.g. cell boundaries) only via the brightness values and not via differences in color, the image objects obtained reflect the morphological structures. The image objects can be combined by combining adjacent pixels with similar brightness values.

The invention furthermore relates to an apparatus for cultivating cells, cell aggregates and tissues including an incubator. The apparatus is characterized in that it furthermore includes a device for objectively characterizing the cells, cell aggregates or tissue as claimed in any of claims 1 to 6. The particularity of the invention consists in that the device for objectively characterizing the cells, cell aggregates or tissue is an integral part of an apparatus for cultivating cells, cell aggregates or tissue. In accordance with a preferred aspect of the invention, this is an apparatus for automatically cultivating cells, cell aggregates and tissue, i.e. an automatic cell culture apparatus with an integrated analysis unit for objectively characterizing the cells, cell aggregates or tissue. Furthermore, the analysis unit should also preferably automatically detect and evaluate morphological parameters during cultivation.

It is possible to include the result of the evaluation in the decision processes of the automation, i.e. to operate the apparatus and the cell cultivation in dependence on the evaluation.

In accordance with a preferred aspect of the invention, the apparatus furthermore includes a manipulator, a transporter for transporting the culture vessels between the incubator, the manipulator and the analysis unit as well as a control unit, by means of which the apparatus can preferably be operated automatically. The invention in accordance with this aspect thus includes a plurality of functional groups, which will be described in detail below:

The detector constitutes a core element of the invention. It serves to monitor the cell population during cultivation. The detector or the above-mentioned analysis unit consists of an image-forming unit as well as a software-assisted image analysis unit with integrated expert knowledge, by means of which the morphological parameters can be detected in their complexity.

As body conditions must be simulated for the cell cultivation of cells, an incubator is integrated in the automatic cell culture apparatus.

The manipulator accomplishes all those process steps automatically, which so far have been performed purely by hand or partly with the aid of machines. These steps include, for instance, sucking off and pipetting solutions, centrifugation, opening the culture vessels, mechanical detachment of cells, etc.

By means of the transporter, which consists of a combination of conveyor belt and robot with gripper arm, the cell culture vessels are moved back and forth between the automatic components detector, incubator and manipulator.

Furthermore, an intelligent control unit is provided, which actuates the individual components of the apparatus and coordinates the activity thereof. The control unit receives the information necessary for the intelligent operation by the detector and the analysis unit, respectively.

In accordance with another aspect of this apparatus, the software-assisted image analysis unit automatically performs complex morphometric image analyses. This unit is in particular characterized in that the expert knowledge can be implemented very flexibly, which is required for detecting and evaluating the complex morphology.

The invention furthermore relates to a method with the features as claimed in claim 10. Advantageous aspects of the method are subject-matter of the sub-claims.

Further details and advantages of the invention will be explained in detail with reference to an embodiment illustrated in the drawing, in which:

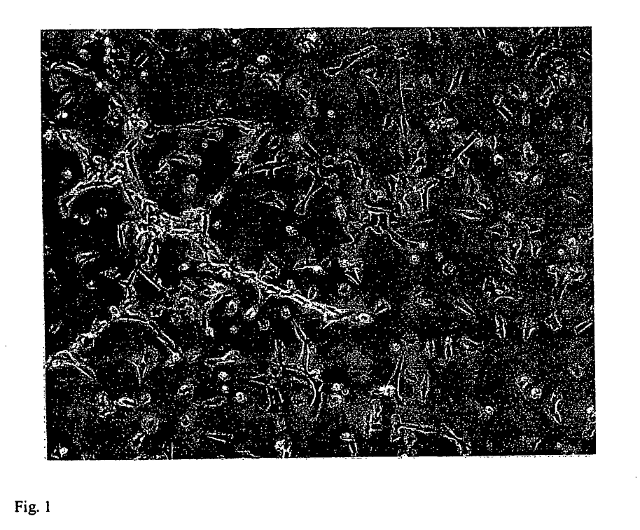

FIGS. 1 to 4: show different exemplary views of cell populations to be characterized,

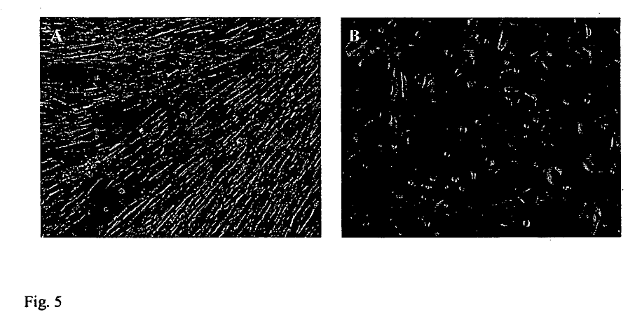

FIG. 5: shows representations of a confluent fibroblast structure (A) and an endothelial cell culture (B),

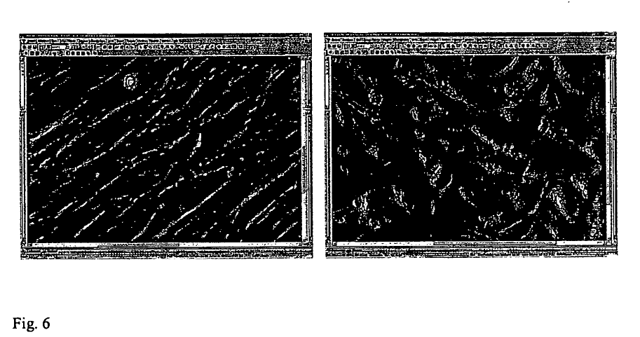

FIG. 6: shows the contour of the image objects (A: fibroblast cells, B: endothelial cells),

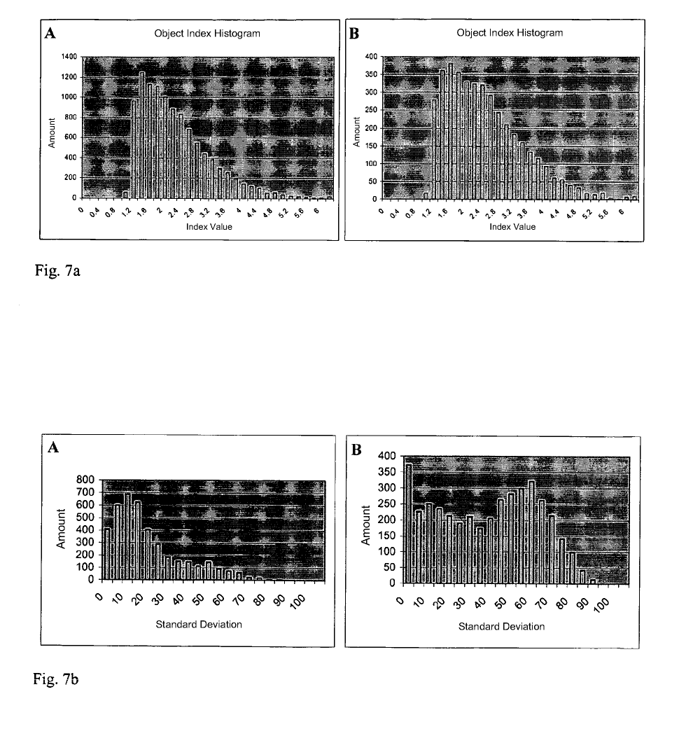

FIG. 7a: shows the frequency distribution of the image object index (circumference/(4×square root of the surface area)) of the image objects as shown in FIG. 5,

FIG. 7b: shows the frequency distribution along the principal axis of the cell populations as shown in FIG. 5.

FIG. 1 shows an exemplary view of a cell culture to be characterized. Conceivable characterization features include: cell population consists of x % roundish and y % oblong cells; the cells carry small vesicles at their edges; the cells partly form into strands.

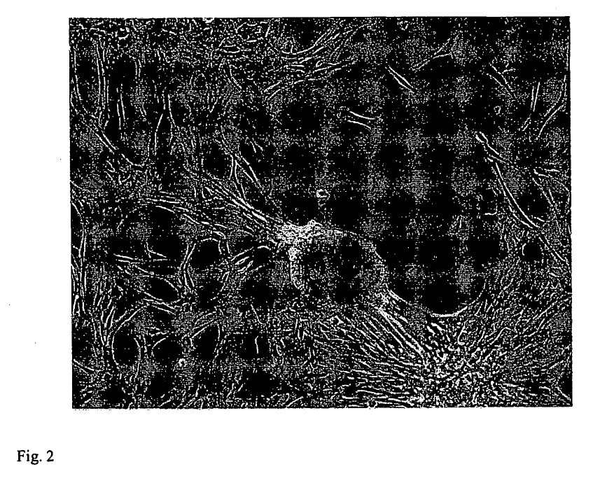

Typical characteristics of the cell structure as shown in FIG. 2 include: cells constitute three-dimensional formations; the cells are more oblong, the cells are less granular.

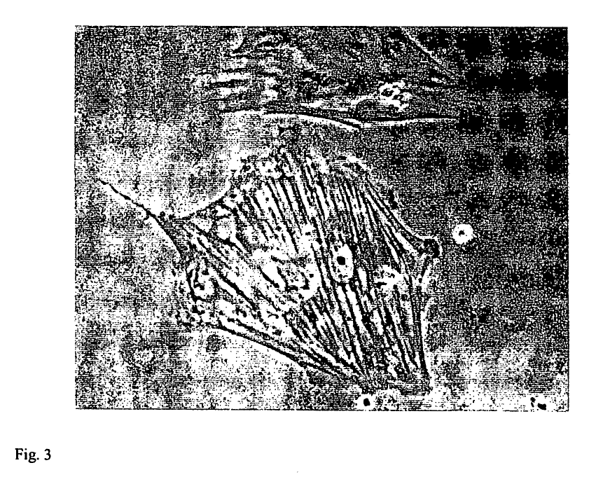

Characteristics of the cells as shown in FIG. 3 include: The cells are characterized by extreme striation (“stress fibers”); the cells have a prominent nucleolus; the cells are flat; the cells are large; the cells have a serrated edge.

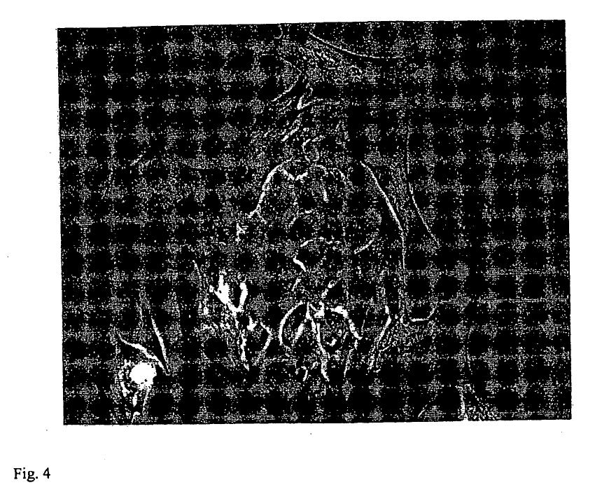

Characteristics of the cells as shown in FIG. 4 include: There are two cell populations, namely large flat cells, lying in a loose aggregate, partly striated, and smaller roundish cells forming a cobblestone-like aggregate.

As will subsequently be described in greater detail, the above-mentioned parameters of the cell populations, which can be detected by means of an image-forming unit, can be used for the characterization thereof.

The principle of the method of the invention and the operation of the apparatus of the invention will be explained in detail with reference to an example given below. The objective is to distinguish a fibroblast culture from an endothelial cell culture by means of a morphological index. The cell cultures can be seen in FIG. 5, wherein FIG. 5A shows the fibroblast culture and FIG. 5B shows the endothelial cell culture. The characterization is effected by means of a morphometric analysis of the cell culture images (phase-contrast images).

There was established a set of analysis rules, according to which adjacent pixels with similar brightness values are combined to form image objects. As the phase-contrast images virtually reveal the morphological structures (e.g. cell boundaries) only by means of the brightness values and not by means of differences in color, the image objects obtained reflect the morphological structures. As can be seen from a look at the appearance of the two cell cultures as shown in FIG. 5, the fibroblast culture is chiefly characterized by oblong morphological structures (spindle-shaped aspect of fibroblasts), whereas the endothelial cell culture has a cobblestone-like aspect (FIG. 5). The Figures mentioned below reveal that this is also reflected in the form of the image objects obtained during computer analysis: In the case of the fibroblast culture, objects with an oblong character can be found to a greater extent, which as compared to the image objects of the endothelial cell culture have a smaller degree of fractality, as can be seen in FIG. 6.

Circumference and surface area (each in pixels) of each image object were now output into an Excel table. Subsequently, an object index was defined as follows: Index=object circumference/(4×square root (object area)).

This formula describes the degree of fractality. The factor ¼ as well as the square root effect a standardization of the image objects, so that objects of different sizes can also be compared with each other (the quotient object circumference/area greatly depends on the size of the objects).

This index was now calculated for each image object. If all object index values of the image objects are now represented in a histogram, there is obtained a kind of characteristic curve for the respective cell culture, as can be taken from FIG. 7a. Characteristic values for describing the course of the histogram include e.g. the mean value, the standard deviation as well as the median. It can be seen that the two curves as shown in FIG. 7a exhibit a different course. The curve for the endothelial cell culture is more bulged. The values for the mean value, the standard deviation as well as the median also are different for the fibroblast and endothelial cell cultures (fibroblasts: mean value 2.17; standard deviation: 0.91; median: 1.96. Endothelial cells: mean value: 2.36; standard deviation: 0.96; median: 2.18).

Said values can thus be used for the objective numerical characterization of the cell culture morphology.

Another characteristic, which distinguishes the fibroblast culture from the endothelial cell culture, is the orientation of the individual image objects. As can be seen in FIG. 6, the principal axes of adjacent image objects are largely parallel in the fibroblast culture, whereas this is much less the case in the endothelial cell culture. This object property can also be described mathematically by corresponding programming work and can be used for characterizing the morphological appearance of the cell cultures (FIG. 7b). The mean value of the distribution shown on the left is 15.97 for the fibroblast cell culture and that of the distribution shown on the right is 36.04 for the endothelial cell culture.

Correspondingly, numerous indices can be defined and be used for characterization. For this purpose, the indices can be arranged e.g. in a two-dimensional array, and the index values can be color-coded. There is thus obtained a colored pattern characteristic for the cell culture.

On the other hand, the individual index values can be combined by arithmetical operations to provide a new index. It would make sense, for instance, to combine the above-defined index of circumference and area (fractality) with the index that describes the parallelism which is characteristic for the image objects of the fibroblast culture, as they both have a relatively low fractality and are arranged in parallel. The new index thus defined has a better distinctiveness as a result of the combination.

Due to the growing sector of economy in the field of methods of cellular therapy, it will be necessary more and more to grow cells under certifiable conditions. This is also true for that field of the pharmaceutical industry in which drugs are tested by means of cell cultures. By comparing the statistically described morphometric fingerprint of a cell culture with the one stored as reference in the file for this type of cell, it is possible to also certify the morphological assessment.

1. An apparatus for characterizing cells, cell aggregates and/or tissue comprising:
an analysis unit which includes means by which morphological parameters of cells, cell aggregates and/or tissue are detected, and which includes means by which the detected morphological parameters are evaluated for the purpose of the objective morphological characterization of the cells, cell aggregates and/or tissue;
a database with reference parameters typical for cells, cell aggregates and/or tissue; and
comparator means by which the detected parameters are compared with the reference parameters.

2. The apparatus as claimed in claim 1, wherein the analysis unit comprises an image-forming unit and an image analysis unit.

3. The apparatus as claimed in claim 1, wherein the analysis unit is configured such that a degree of confluence, a cell morphology as a measure for the quality of the cell culture, a proliferation behavior, a presence of microorganisms and/or a cell differentiation can be detected and evaluated.

4. The apparatus as claimed in claim 1, wherein the analysis unit includes means for the statistical evaluation of the detected morphological parameters.

5. (canceled)

6. The apparatus as claimed in claim 1, wherein the analysis unit includes means by which adjacent pixels of a detected image with similar brightness values are combined to one image object.

7. An apparatus for cultivating cells, cell aggregates and/or tissue comprising:
an incubator, and
a device for objectively characterizing cells, cell aggregates and/or tissue, the device an analysis unit which includes means by which morphological parameters of cells, cell aggregates and/or tissue are detected, and which includes means by which the detected morphological parameters are evaluated for the purpose of the objective morphological characterization of the cells, cell aggregates and/or tissue; a database with reference parameters typical for cells, cell aggregates and/or tissue; and comparator means by which the detected parameters are compared with the reference parameters.

8. The apparatus for cultivating cells as claimed in claim 7, wherein the apparatus furthermore includes a manipulator, a transporter for transporting one or more cell culture vessels between the incubator, the manipulator and the analysis unit as well as a control unit for operating the apparatus.

9. The apparatus as claimed in claim 8, wherein the control unit is configured such that the apparatus is operated automatically.

10. A method for characterizing cells, cell aggregates and/or tissue, comprising:
detecting morphological parameters of the cells, cell aggregates and/or tissue; and
then evaluating the detected morphological parameters in a manner suitable for the objective morphological characterization of the cells, cell aggregates and/or tissue based on the evaluation, the detected parameters being compared with reference parameters typical for cells, cell aggregates and/or tissue, which are stored in a database.

11. The method as claimed in claim 10, wherein statistical values characteristic for the cells, cell aggregates and/or tissue are determined from the detected parameters.

12. The method as claimed in claim 11, wherein the statistical values are compared with values from a reference database which contains values characteristic for cells, cell aggregates and/or tissue.

13. The method as claimed in claim 10, wherein the method furthermore includes the cultivation of cells, cell aggregates and/or tissue.

14. The method as claimed in claim 13, wherein the cell cultivation is performed in dependence on the evaluated parameters.

15. The method as claimed in claim 13, wherein the detection and evaluation of the morphological parameters as well as the cultivation of cells are effected automatically.
